(19) United States
(12) Patent Application Publication
(10) Pub. No.: US 2004/0127402 A1
(43) Pub. Date: Jul. 1, 2004

(54) Injectible composition and method for treating degenerative animal joints

(76) Inventor: Vijay B. Vad, New York, NY (US)

Correspondence Address: WHYTE HIRSCHBOECK DUDEK S C, 555 EAST WELLS STREET, SUITE 1900, MILWAUKEE, WI, 53202, US

(21) Appl. No.: 10/330,766

(22) Filed: Dec. 27, 2002

(51) Int. Cl. A61K 38/31; A61K 31/737; A61K 31/728

(52) U.S. Cl. 514/9; 514/54; 514/62

(57) ABSTRACT

An injectible, slow-release composition for treating animal, particularly mammalian and especially human, joints is described. The composition comprises (i) a lubricant, (ii) an anti-inflammatory agent, (iii) a growth hormone, and (iv) a slow-release polymer. The composition can be administered to any joint, e.g., spinal, knee, elbow, shoulder, hip, ankle, wrist, digit, etc., that is experiencing pain and/or the degeneration of connective tissue as a result of disease (e.g., osteoarthritis), stress, strain and/or other afflictions or ailments.

### BACKGROUND OF THE INVENTION

In one aspect, this invention relates to a composition for treating the joints of humans/animals. In another aspect, this invention relates to a composition comprising a joint lubricant, an anti-inflammatory agent, a growth hormone/growth factor and a slow-release agent while in yet another aspect, this invention relates to a method of using the composition to treat the human/animal joint in a degenerative state.

The connective tissue found in animal joints, particularly mammalian joints and especially human joints, is constantly subject to stress, strain and disease, resulting in afflictions, such as osteoarthritis, joint inflammation and stiffness. Such afflictions may be painful and may cause joint degeneration, e.g., damage to or loss of connective tissue such as cartilage.

The treatment of such afflictions is often problematic and difficult to sustain. As a result, treatment is commonly directed at controlling the symptoms of the afflictions and not their causes, regardless of the degeneration of the joint.

Presently, several injectible compositions such as steroids, hyaluronic acid, glucosamine, chondroitin sulfate, and growth hormones or growth factors are used for the treatment of such afflictions. However, although such injectible compositions have shown good results, the effect of such compositions is short-lived in duration and provides limited efficacy for treatment of the joint disease or aliment.

Steroid injections have been used commercially for numerous years; Hollander J L: Intra-Articular Hydrocortisone in Arthritis and Allied Conditions. Steroid injections have shown good short-term efficacy with a duration of up to approximately eight weeks, but without any chondroprotective effects; J. Bones Joint Surg. Am. 35:983-990, 1953.

Hyaluronic acid has been used as well, but it has demonstrated to be superior to oral medications for only up to six months in duration; Osteoarthritis Cartilage 3:213-226, 1995. Additionally, using a joint lavage prior to hyaluronic acid therapy has been shown as possibly having positive benefits for up to one year in duration. Hyaluronic acid has been shown to have mild chondroprotective effect in animal studies; Marshall K W: The Current Status of Hylan Therapy for the Treatment of Osteoarthritis. Today's Therapeutic Trends 15: 99-108, 1997. One drawback to hyaluronic acid therapy is that this composition has a very low success rate with treating advanced osteoarthritis and is relatively short-lived in duration.

Glucosamine and chondroitin sulfate injectibles possess superior pain reduction properties with a short-lived duration of only up to four weeks compared to controls. Growth hormones have been shown to alleviate pain and increase function of the joint without unwanted side effects. One major drawback of such compositions is the lack of long-term efficacy.

Connective tissue is naturally equipped to repair itself by producing collagen (a primary component of connective tissue such as cartilage) and proteoglycans (another major component of connective tissue such as cartilage). The building blocks for collagen are amino acids, primarily proline, glycine and lysine. Proteoglycans provide the framework for collagen formation and also hold water in order to provide flexibility, resiliency and resistance to compression.

Glycosaminoglycans and collagen are the chief structural elements of all connective tissues. Their synthesis is essential for proper maintenance and repair of connective tissues. The introduction of glucosamine has been demonstrated to increase the synthesis of collagen and glycosaminoglycans in fibroblasts, which is the first step in the repair of connective tissues.

Glucosamine is the main building block of connective tissue and may be provided either through the enzymatic conversion of glucose or through diet or external administration. Glucosamine may be converted into proteoglycans upon incorporation of glucosamine into glycosaminoglycans. Glucosamine has also exhibited reproducible improvement in cartilage integrity in humans with osteoarthritis.

Chondroitin sulfate is a glycosaminoglycan that provides a further substraight for the synthesis of proteoglycans. The provision of the chondroitin in its salt (sulfate) form facilitates its delivery and uptake by humans and animals in the production of connective tissue. The sulfate portion of chondroitin sulfate is also available for use in catalyzing the conversation of glucosamine to glycosaminoglycans.

Chondroitin may be more efficacious than glycosamine for injury rehabilitation. Chondroitin sulfate also acts to inhibit the degradative enzymes that break down connective tissues. Chondroitin sulfate promotes the maintenance of healthy connective tissues. The ability of chondroitin sulfate to block degradation is one of its important functions.

While these materials often relieve the pain and swelling associated with these afflictions, their effectiveness diminishes over relatively short period of time.

### SUMMARY OF THE INVENTION

The present invention is an injectible, slow-release composition for treating animal, particularly mammalian and especially human, joints. The composition comprises (i) a lubricant, (ii) an anti-inflammatory agent, (iii) a growth hormone/growth factors and (iv) a slow-release polymer. The composition can be administered to any joint, e.g., spinal, knee, elbow, shoulder, hip, ankle, wrist, digit, etc., that is experiencing pain and/or the degeneration of connective tissue as a result of disease (e.g., osteoarthritis), stress, strain and/or other afflictions or ailments. The composition provides a synergistic blend of properties to a joint in a degenerate state such that a recipient of a therapeutically effective dose of the composition will experience a greater clinical reduction in pain with greater chondroprotection than a recipient of a therapeutically effective dose of each component of the composition independent of one another.

In one embodiment of the invention, the lubricant comprises a hyaluronic acid, the anti-inflammatory agent comprises at least one of glucosamine and chondroitin sulfate, the growth hormone comprises somatostatin or growth factor such as IGF, and the slow-release polymer comprises at least one of polylactic acid and polylactic-coglycolic acid. Sufficient composition, i.e., a therapeutically effective quantity, is injected into the joint using a standard syringe so as to provide at least one of a decrease in pain and a retardation of connective tissue over an extended period of time, e.g., at least one month.

The composition is produced by blending the components into a relatively homogeneous mixture using gentle agitation under ambient conditions. The components can be mixed with one another in any sequence.

### DETAILED DESCRIPTION OF THE INVENTION

The lubricant component of the composition can comprises any material that will provide lubricity, pain reduction and/or at least mild chondroprotective properties to an animal joint. Typically and preferably, the lubricant comprises hyraluronic acid. Hyraluronic acid is a known polymer of acetylglycosamine and glucuronic acid, and it is known component of synovial fluids.

The anti-inflammatory agent or component of the composition can comprise any material that will reduce the inflammation, swelling and/or pain of a joint that is the result of disease or injury. Typically, the anti-inflammatory agent comprises at least one of chondroitin sulfate and glucosamine. Chondroitin sulfate, is a well know compound and a major constituent of the cartilaginous tissue of the human and other animal bodies. Glucosamine, CH₂OH(CHOH)₃CHNH₂CHO, is also a well known compound, and it is usually available in the D(+) form.

The chondroitin sulfate and/or glucosamine can be used alone or in combination with one another and/or in combination with one or more other materials, e.g., another anti-inflammatory agent or an inert material, e.g., a dye, stabilizer, etc. If the anti-inflammatory agent comprises chondroitin sulfate in combination with glucosamine, then typically the chondroitin sulfate comprises at least about 5, preferably at least about 10 and more preferably at least about 15, percent of the total weight of the agent. Typically, the maximum amount of chondroitin sulfate in the anti-inflammatory agent does not exceed about 50, preferably about 40 and more preferably about 30, percent by weight of the total weight of the agent. The glucosamine typically comprises at least about 50, preferably at least about 60 and more preferably at least about 80, percent by weight of the anti-inflammatory agent. Typically, the maximum amount of glucosamine in the agent does not exceed about 95, preferably about 90 and more preferably about 85, percent by weight of the total weight of the agent.

The growth hormone component of the composition can comprise any hormone or factor that will promote connective tissue health and/or growth. Typically and preferably, the growth hormone component comprises somatostatin, a well know hormone associated with general body growth and carbohydrate and lipid metabolism. The growth hormone component can comprise somatostatin alone or somatostatin in combination with one or more other materials, e.g., another growth hormone or growth factors such as insulin growth factor or fibrin growth factor. If the growth hormone comprises somatostatin in combination with one or more other materials, then typically the somatostatin comprises at least about 20, preferably at least about 25 and more preferably at least about 50 percent of the total weight of the growth hormone component.

The slow-release polymer component of the composition can comprise any polymer that will control the release of the other components of the composition to the joint over an extended time period. As used in this disclosure, “extended time period” means at least about one, preferably at least about three months and more preferably at least about six months measured from the time that the composition is injected into the joint until the therapeutic effect of the composition is essentially spent. This period of time is, of course, a function of many variables including but not limited to the nature (both in terms of structure and health) of the joint, the components and their relative amounts in the composition, the dosage or amount of composition injected into the joint, and the like. One skilled in the art of joint therapy can match these variables to optimize the therapeutic effect over time of the composition on the joint.

Typically and preferably, the slow-release polymer component comprises at least one of polylactic acid and polylactic-coglycolic acid, both well know polymers. Either polymer can be used alone or in combination with one another and/or in combination with one or more other materials, e.g., another slow-release polymer or an inert material, e.g., a dye, stabilizer, anti-oxidant, etc. If the slow-release polymer comprises polylactic acid in combination with polylactic-coglycolic acid, then the weight ratio of one to the other can vary from 1:99 to 99:1 although preferably, the weight ratio of polylactic acid to polylactic-coglycolic acid is between about 1:5 and about 1:90. If the slow-release polymer comprises one or more other materials in combination with one or both of the polylactic acid and polylactic-coglycolic acid, then the total weight of the polylactic acid and/or polylactic-coglycolic acid is typically at least about 30, preferably at least about 40 and more preferably at least about 80, percent of the total weight of the slow-release polymer component.

The composition of the invention can be prepared in any convenient manner. Typically, each component is prepared separately, and the components are combined with one another. The components can be added in any order, but typically the components that constitute a minor proportion of the composition, relative to the other components, are added to the component or components that constitute a major proportion of the composition. The components are usually blended with one another under ambient conditions and with gentle agitation to form an essentially homogeneous mixture. The composition can contain materials or components other than the lubricant, anti-inflammatory agent, growth hormone and slow-release polymer, e.g., preservatives, diluents, dyes, etc.

The relative amounts of each component in the composition can vary to the nature and condition of the joint. Representative compositions comprise between (i) about 0.01 and about 1, preferably between about 0.02 and about 0.08 and more preferably between about 0.03 and about 0.08 weight percent joint lubricant, (ii) about 50 and about 80, preferably between about 55 and about 75 and more preferably between about 50 and about 70 weight percent anti-inflammatory agent, (iii) about 0.001 and about 0.050, preferably between about 0.010 and about 0.030 and more preferably between about 0.014 and about 0.020 weight percent growth hormone or growth factor, and (iv) about 20 and about 50, preferably between about 25 and about 45 and more preferably between about 30 and about 40 weight percent slow-release polymer, based on the total weight of the composition. If one or more other components are present in the composition, then these other components typically comprise in the aggregate between about 1 and about 20, preferably between about 5 and about 15 and more preferably between about 10 and about 15, weight percent of the composition. Often, the slow-release polymer is a major component of the composition and as such, the composition often has a gel or viscous liquid consistency.

For treatment of a human joint with a composition comprising hyraluronic acid, glucosamine, chondroitin sulfate, somatostatin and polylactic acid, typical dosages for each component are about 2 to about 8 milligrams (mg) of hyraluronic acid with 2-4 mg as more preferable, about 2000 to about 8000 milligrams of glucosamine with 2000 to 5000 as more preferable, about 750 to about 2000 milligrams of chondroitin sulfate with 750 mg to 1250 mg as more preferable, about 750 micrograms (mcg) to about 2000 mcg of somatostatin/growth factors with 750 mcg to 1200 mcg as more preferable, and about 2 to about 4 grams of polylactic acid with 2 to 2.5 grams as more preferable.

The compositions of this invention are used in the same manner as known injectibles for joints. The composition is formulated, loaded or drawn into a syringe, the joint prepared to receive the injection, e.g., the skin cleansed and/or anesthetized, the joint opened or closed, etc., and the composition injected into the joint. The composition can be injected in a single dosage, or delivered in multiple dosages either in one session or over multiple sessions. If multiple injections are used to deliver the composition to the joint, the injections can be to the same or different locations of the joint, e.g., the left and right side of a knee joint. The amount or dosage of composition injected into the joint will also vary with the nature of the joint, but the typical minimum amount of composition injected into the joint is a therapeutic amount. Usually a single dosage is between about 1 and about 5, preferably between about 1 and about 2 and more preferably between about 1.5 and about 2, cubic centimeters (cc).

While the compositions are useful for any animal joint with degenerative connective tissue, the compositions of this invention are particularly useful for degenerative mammalian, especially human, joints. Representative animals other than humans include dogs, cats, horses, birds, and the like. Representative degenerative joints include spinal, knee, hip, elbow, wrist, shoulder, ankle and digit, suffering from osteoarthritis. Proper use of the composition of this invention can reduce the pain and retard the progression of osteoarthritis and this, in turn, can save a human or animal joint or disc replacements or lumbar fusion. This, in turn, can save considerable health care costs.

Although the invention has been described in considerable detail through the proceeding embodiments, this detail is for the purpose of illustration. Many variations and modifications can be made without departing from the spirit and scope of the invention as described in the appended claims. All United States patents and allowed United States patent applications cited above are incorporated herein by reference.

What is claimed is:

1. An injectible, slow-release composition for treating an animal joint, the composition comprising (i) a joint lubricant, (ii) an anti-inflammatory agent, (iii) a growth hormone, and (iv) a slow-release polymer.

2. The composition of claim 1 in which the joint lubricant is hyraluronic acid.

3. The composition of claim 1 in which the anti-inflammatory agent is at least one of glucosamine and chondroitin sulfate.

4. The composition of claim 1 in which the growth hormone is somatostatin.

5. The composition of claim 1 in which the slow-release polymer is at least one of polylactic acid and polylactic-coglycolic acid.

6. The composition of claim 2 in which the anti-inflammatory agent is at least one of glucosamine and chondroitin sulfate.

7. The composition of claim 2 in which the growth hormone is somatostatin.

8. The composition of claim 2 in which the slow-release polymer is at least one of polylactic acid and polylactic-coglycolic acid.

9. The composition of claim 6 in which the growth hormone is somatostatin.

10. The composition of claim 6 in which the slow-release polymer is at least one of polylactic acid and polylactic-coglycolic acid.

11. The composition of claim 9 in which the slow-release polymer is at least one of polylactic acid and polylactic-coglycolic acid.

12. The composition of claim 11 comprising about 2 to about 4 milligrams of hyraluronic acid, about 2000 to about 3000 milligrams of glucosamine, about 750 to about 1200 milligrams of chondroitin sulfate, about 750 micrograms to about 2000 micrograms of somatostatin or growth factors, and about 1 to about 2 grams of polylactic acid.

13. A method of treating a degenerative animal joint, the method comprising injecting into the joint a therapeutically effective amount of the composition of claim 1.

14. A method of treating a degenerative animal joint, the method comprising injecting into the joint a therapeutically effective amount of the composition of claim 11.

15. A method of treating a degenerative animal joint, the method comprising injecting into the joint a therapeutically effective amount of the composition of claim 12.

16. The method of claim 13 in which the joint is a mammalian joint.

17. The method of claim 13 in which the joint is a human joint.

18. The method of claim 15 in which the joint is a mammalian joint.

19. The method of claim 15 in which the joint is a human joint.

20. The method of claim 19 in which the joint is a spinal, ankle, knee, hip, shoulder, elbow, wrist or digit joint.
